SEQUENCE LISTING (17 sequences)

SEQ ID NO: 1 - protein, 641 aa, Epstein-Barr virus
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 2 - protein, 428 aa, synthetic construct
  REGION 1..428: note = EBNA1_A
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGARGRGGSGGGGSGGAGGGGSGGAGGSGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 3 - protein, 389 aa, synthetic construct
  REGION 1..389: note = Artificial
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKGGGGAGGGGSGGGGAGGGGSGGGGAGGGGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 4 - protein, 213 aa, synthetic construct
  REGION 1..213: note = Artificial
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 450 aa, synthetic construct
  REGION 1..450: note = Artificial
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
  REGION 1..214: note = Herceptin light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, synthetic construct
  REGION 1..450: note = Herceptin heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, synthetic construct
  REGION 1..214: note = Emicizumab light chain
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, synthetic construct
  REGION 1..448: note = Emicizumab heavy chain-1
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 10 - protein, 444 aa, synthetic construct
  REGION 1..444: note = Emicizumab heavy chain-2
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 11 - protein, 275 aa, synthetic construct
  REGION 1..275: note = Dulaglutide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 30 aa, synthetic construct
  REGION 1..30: note = Artificial
RGRGGSGGGGSGGAGGGGSGGAGGSGGSGG

SEQ ID NO: 13 - protein, 120 aa, synthetic construct
  REGION 1..120: note = Artificial
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKGGGGAGGGGSGGGGAGGGGSGGGGAGGG

SEQ ID NO: 14 - protein, 28 aa, synthetic construct
  REGION 1..28: note = Short linker in EBNA1_B
GGGGAGGGGSGGGGAGGGGSGGGGAGGG

SEQ ID NO: 15 - protein, 92 aa, synthetic construct
  REGION 1..92: note = AT hook motif in EBNA1_B
MSESSSKSSQPLASKQEKDGTEKRGRGRPRKQPPVSPGTALVGSQKEPSEVPTPKRPRGRPKGSKNKGAAKTRKTTTTPGRKPRGRPKKLEK

SEQ ID NO: 16 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0250327 A1
(43) Pub. Date: Aug. 27, 2026

(54) FUNCTION-ENHANCED ENGINEERED EBNA1 FOR PROTEIN EXPRESSION IN MAMMALIAN CELLS

(71) Applicant: WuXi Biologics Ireland Limited, Co Louth (IE)

(72) Inventors: Shanhui Liao, Shanghai (CN); Huihui Lu, Shanghai (CN); Qian Guo, Shanghai (CN); Xiaoyue Chen, Shanghai (CN); Jiexing Cai, Shanghai (CN); Weichang Zhou, Shanghai (CN)

(21) Appl. No.: 18/995,498

(22) PCT Filed: Jul. 28, 2023
(86) PCT No.: PCT/CN2023/109708

§ 371 Date: Jan. 16, 2025

(30) Foreign Application Priority Data
Jul. 29, 2022 (WO) PCT/CN2022/109012

(51) Int. Cl. C07K 14/005; C07K 14/605; C07K 16/24; C07K 16/28; C07K 16/32; C07K 16/36; C12N 15/85

(52) U.S. Cl. CPC C07K 14/005; C07K 14/605; C07K 16/241; C07K 16/2809; C07K 16/32; C07K 16/36; C12N 15/85; C07K 2317/20; C07K 2317/21; C07K 2317/24; C12N 2710/16222

(57) ABSTRACT

Provided herein are engineered Epstein-Barr virus nuclear antigen 1 (EBNA1), coding molecules thereof, vectors and mammalian cell expression systems comprising the same, and polypeptide of interest recombinantly produced by the foregoing. Also provided are methods for the preparation of the engineered EBNA1s, coding molecules thereof, vectors and mammalian cell expression systems and methods for using the same in recombinant expression.

### TECHNICAL FIELD

The present disclosure concerns the field of genetic engineering and recombinant expression technologies. It inter alia pertains to altered mammalian cell expression systems, for example CHO and HEK293 cell expression systems, which comprise vectors or stable cell lines expressing function-enhanced engineered Epstein-Barr virus nuclear antigen 1 (EBNA1 (s)) to improve transient gene expression.

### BACKGROUND OF THE INVENTION

Since separating protein products from natural sources are limited, various techniques and expression systems have been developed for recombinant protein expression for such as clinical and research usage. Comparing to the prokaryotic expression system, mammalian cell expression system displays huge advantages in many aspects, including protein post-translational modifications, protein folding quality and high yield protein production (Nagesh and Ambuj, 2019). Thus, mammalian cell expression system is the most dominant system for biopharmaceutical production, such as production of antibodies, cytokines, growth factors and so on (Jianwei, 2012). Transient expression by transfecting cells with plasmids is a popular technique for rapid recombinant protein production in mammalian cells, and numbers of efforts including improving transfection efficiency, vector engineering and cell engineering, have been put in to enhance protein production titer (Kishwar, 2013).

For transient expression in mammalian cells, how to maintain a high level of plasmid concentration in nucleus is the major concern for high level recombinant protein expression. In the nature, some viruses, such as Epstein-Barr virus (EBV), employ different strategies to maintain their genome copy number during host cell proliferation. Epstein-Barr virus nuclear antigen 1 (EBNA1) protein plays key roles in virus genome replication and maintains viral episomes in infected host cells by associating episomes to host chromosomes during host cell replication (Wolfgang and Bill, 2013). EBNA1 protein contains two DNA binding regions: one is an N-terminal AT-hook responsible for host chromosome association, and another one is a C-terminal OriP DNA binding domain responsible for viral episomes tethering (John et al. 2004 and Tohru et al. 2004). The unique structure of EBNA1 ensures efficient propagation of virus genomes to host daughter cells. Besides episomal DNA, OriP containing plasmids can also be maintained at a relative high level by EBNA1 after transfection (Ann and Bill, 1995). Transient mammalian expression systems based on EBNA1-OriP interaction have been developed and approved to significantly improve recombinant protein production (Francoise et al., 1998 and Olalekan et al., 2014).

It has been reported that foreign protein productivity in mammalian cell is positively correlated with EBNA-1 expression level (Joo-Hyoung et al., 2017). Thus, enhanced EBNA1 protein expression level or enhanced EBNA1-chromosome association may further improve recombinant protein expression level. There is still a great need for recombinant mammalian cell expression systems with improved transient gene expression.

### SUMMARY OF THE INVENTION

Disclosed herein are novel engineered EBNA1 (s) and coding molecules thereof, vectors and mammalian cell expression systems comprising the same having improved transient gene expression, and the preparation and use of the foregoing.

According to one aspect, the present disclosure provides an engineered EBNA1, wherein as compared to wild-type EBNA1, the original G-A rich region is engineered in the engineered EBNA1.

In some embodiments, in the engineered EBNA1 (a) the original G-A rich region is removed; (b) the original G-A rich region is replaced by a linker; and/or (c) the N-terminus fragment comprising the original G-A rich region (such as residue 1 to residue 336 at the N-terminus) of wild-type EBNA1 is replaced by an AT hook motif capable of binding to a chromosome at mitotic phase, such as an AT hook motif (such as residue 1 to residue 92) from human HMG-I/Y.

According to a further aspect, the present disclosure provides an isolated polynucleotide molecule encoding the engineered EBNA1 (s) disclosed herein.

According to a further aspect, the present disclosure provides a vector comprising one or more of the polynucleotide molecule disclosed herein.

According to a further aspect, the present disclosure provides an isolated mammalian cell suitable for recombinant expression of a polypeptide of interest, wherein the mammalian cell comprises an engineered EBNA1, wherein the engineered EBNA1 has enhanced function in recombinant expression as compared to wild-type EBNA1 (such as a wild-type EBNA1 comprises an amino acid sequence of SEQ ID NO: 1), and wherein the original G-A rich region of wild-type EBNA1 is removed or replaced in the engineered EBNA1.

According to a further aspect, the present disclosure provides a method for producing a mammalian cell as disclosed herein, comprising introducing into a mammalian cell the polynucleotide molecule encoding an engineered EBNA1 disclosed herein.

According to a further aspect, the present disclosure provides a method for recombinantly producing or increasing the recombinant expression yield of a polypeptide of interest, comprising
(a) culturing a mammalian cell disclosed herein under conditions that allow for the expression of the polypeptide of interest;
(b) isolating the polypeptide of interest; and
(c) optionally, processing the isolated polypeptide of interest.

According to a further aspect, the present disclosure provides a product for use in the recombinant expression of a polypeptide of interest comprising the isolated polypeptide disclosed herein, the polynucleotide molecule disclosed herein, the vector disclosed herein, and/or the mammalian cell disclosed herein.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Structural feature of wild-type EBNA1 and ENBA1 variants. The wild type EBNA1 contains a long G-A rich region flanked by two Basic regions (“Basic”). The two basic region contains AT hook motifs, which are essential for chromosome association. The Ori-DNA binding domain directly binds to OriP-DNA, and tethers OriP-containing plasmids or episomes to chromosome. The nuclear localization signal (NLS) is responsible for nuclear transport of EBNA1 proteins. EBNA1_A and EBNA1_B are engineered EBNA1 (s). EBNA1_A contains a designed linker to replace the original G-A rich region, and EBNA1_B contains an AT hook motif from human HMG-I/Y and a short linker to replace the whole N-terminal of EBNA1.

FIG. 2: Therapeutic antibody or Fc-fusion protein drugs, including OKT3 (monoclonal antibody), Herceptin (monoclonal antibody), Emicizumab (Bispecific antibody) and Dulaglutide (Fc-fusion protein), are selected as test molecules. Effects of EBNA1 and engineered EBNA1s on transient expression of the test molecules were investigated in CHO cell transient expression system. Plasmids expressing test molecules were co-transfected with or without plasmids expressing wt EBNA1 or engineered EBNA1s (EBNA1_A or EBNA1_B), respectively. Significant difference was determined by t-test. *indicates p value <0.05 with significant difference.

FIG. 3: Therapeutic antibody or Fc-fusion protein drugs, including OKT3 (monoclonal antibody), Herceptin (monoclonal antibody), Emicizumab (Bispecific antibody) and Dulaglutide (Fc-fusion protein), are selected as test molecules. Effects of EBNA1 and engineered EBNA1s on transient expression of the test molecules were evaluated in HEK293 cell transient expression system. Plasmids expressing test molecules were co-transfected with or without plasmids expressing engineered EBNA1s respectively. Significant difference was determined by t-test. *indicates p value <0.05 with significant difference.

FIG. 4: Percentage of viable CHO cells expressing different test molecules was measured to investigate the cytotoxic effects of engineered EBNA1s.

FIG. 5: Percentage of viable HEK293 cells expressing different test molecules was measured to investigate the cytotoxic effects of engineered EBNA1s.

FIG. 6: Average cell productivity of CHO cell for test molecules was calculated based on the titer, cell number and viable cell density. Effects of engineered EBNA1s on transient expression were investigated. Significant difference was determined by t-test. *indicates p value <0.05 with significant difference.

FIG. 7: Average cell productivity of HEK293 cell for test molecules was calculated based on the titer, cell number and viable cell density. Effects of engineered EBNA1s on transient expression were investigated.

FIG. 8: Final product quality of OKT3 with/without co-transfection of engineered EBNA1s was analyzed by SDS-PAGE. Left panel indicated OKT3 protein expressed in CHO cells, and the right panel indicated OKT3 protein expressed in HEK293 cells. NR: non-reduction; R: reduction; Sample reduction was performed by adding 2 mM DTT.

FIG. 9: Final product quality of Herceptin with/without co-transfection of engineered EBNA1s was analyzed by SDS-PAGE. Left panel indicated Herceptin protein expressed in CHO cells, and the right panel indicated Herceptin protein expressed in HEK293 cells. NR: non-reduction; R: reduction; Sample reduction was performed by adding 2 mM DTT.

FIG. 10: Final product quality of Emicizumab with/without co-transfection of engineered EBNA1s was analyzed by SDS-PAGE. Left panel indicated Emicizumab protein expressed in CHO cells, and the right panel indicated Herceptin protein expressed in HEK293 cells. NR: non-reduction; R: reduction; Sample reduction was performed by adding 2 mM DTT.

FIG. 11: Final product quality of Dulaglutide with/without co-transfection of engineered EBNA1s was analyzed by SDS-PAGE. Left panel indicated Dulaglutide protein expressed in CHO cells, and the right panel indicated Dulaglutide protein expressed in HEK293 cells. NR: non-reduction; R: reduction; Sample reduction was performed by adding 2 mM DTT.

FIG. 12: plasmid map of pWX4.1. pWX4.1 contains an OriP element to maintain EBNA1's function, an ampicillin resistance gene (Amp) for plasmid selection, a pUC on element for plasmid amplification in Escherichia coli, a cytomegalovirus (CMV) promoter for gene transcription in host cells, a Herpes simplex virus (HSV) thymidine kinase polyadenylation signal (TKpA) element for RNA termination and polyadenylation and a multiple clone site for insertion of interest gene for production.

FIG. 13: plasmid map of pWX039. pWX039 contains an ampicillin resistance gene (Amp) for plasmid selection, a pUC on element for plasmid amplification in Escherichia coli, a WXRE1 regulatory element and an cytomegalovirus (CMV) promoter for gene transcription in host cells, a Herpes simplex virus (HSV) thymidine kinase polyadenylation signal (TKpA) element for RNA termination and polyadenylation, an internal ribosomal entry site (IRES) from encephalomyocarditis virus (EMCV) for anti-zeocin protein expression and a multiple clone site for insertion of interest gene for production.

FIG. 14: Investigation of protein expression by stable CHO cell line expressing engineered EBNA1 derivate (EBNA1_B). Adalimumab was selected to express in different stable cell line expressing no EBNA1, wild type EBNA1 and engineered EBNA1 respectively. The expression titer on Day 4 and Day 7 for each expression was measured. The stable cell line expressing engineered EBNA1 displayed significant advantage (about 1.8-fold increase of titer) over cell line expressing wild type EBNA1 (indicated by *).

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Improving recombinant protein expression titer is one of the most critical point for cost saving in biopharmaceutical production. Expressing wild-type EBNA1 protein in mammalian cells has been approved to significantly improve yield of many recombinant proteins by transient transfection. GA-rich linker region and basic region of EBNA1 are essential parts of EBNA1. To enhance EBNA1's function in recombinant protein production, we engineered EBNA1 by replacing the G-A rich linker and AT hook motifs, and generated two engineered EBNA1s (for example, EBNA1_A and EBNA1_B). Comparing to wild-type EBNA1, function-enhanced engineered EBNA1s significantly enhance protein production in mammalian cells.

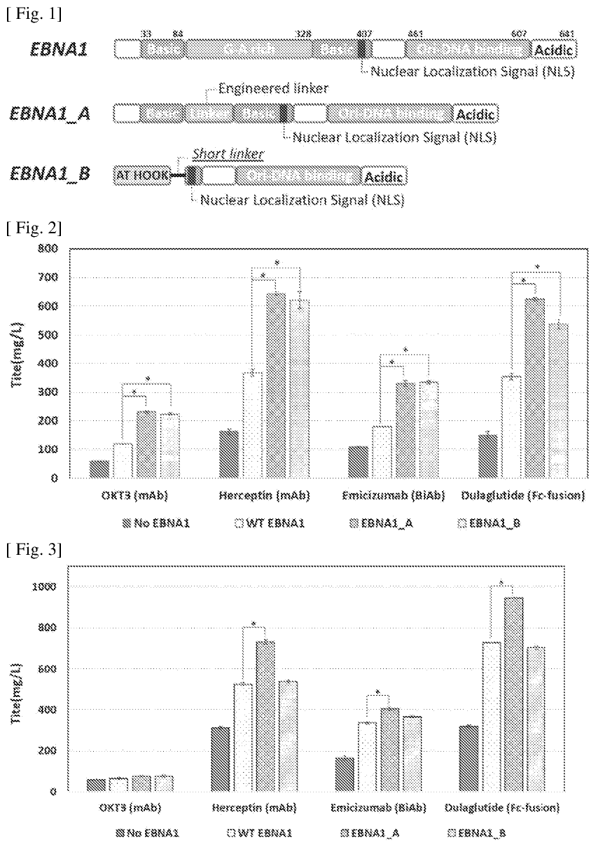

In particular, EBNA1 is a virus protein essential for EBV genome integrity in host cell. Its role in tethering virus genome to host chromosome through OriP element has been widely employed to enhance transient expression in mammalian cells. Based on previous researches, EBNA1 protein contains N-terminal chromosome association domain, a G-A rich region and a C-terminal OriP binding domain (FIG. 1 (top), illustrating the domain architecture of EBNA1).

We designed a molecule named EBNA1_A in which a high translational efficient linker (amino acid sequence “RGRGGSGGGGSGGAGGGGSGGAGGSGGSGG”, SEQ ID NO: 12) was employed to replace the original G-A rich region (region range from 93-325 of EBNA1) for better EBNA1 performance by increasing translation efficiency of EBNA1 (FIG. 1 (middle)).

Enhancement of chromosome association of EBNA1 is another aspect for optimizing its function. EBNA1 possesses two basic regions, in which AT hook domains are responsible for chromosome binding. Some mammalian HMG proteins are specific associate to chromosomes by specific AT hook domain, thus we chosen AT hook domain of HMG-I/HMG-Y to replace the original chromosome binding domain of EBNA1.

We engineered another molecule named EBNA1_B (FIG. 1 bottom) by combining AT hook domain from HMG-I/HMG-Y and a short linker (protein sequence

SEQ ID NO: 13
“MSESSSKSSQPLASKQEKDGTEKRGRGRPRKQPPVSPGTALVG
SQKEPSEVPTPKRPRGRPKGSKNKGAAKTRKTTTTPGRKPRGRPKKLE
KGGGGAGGGGSGGGGAGGGGSGGGGAGGG”,, the underlined
part is the short linker)

and replacing the whole N-terminal 372 residues of wild-type EBNA1.

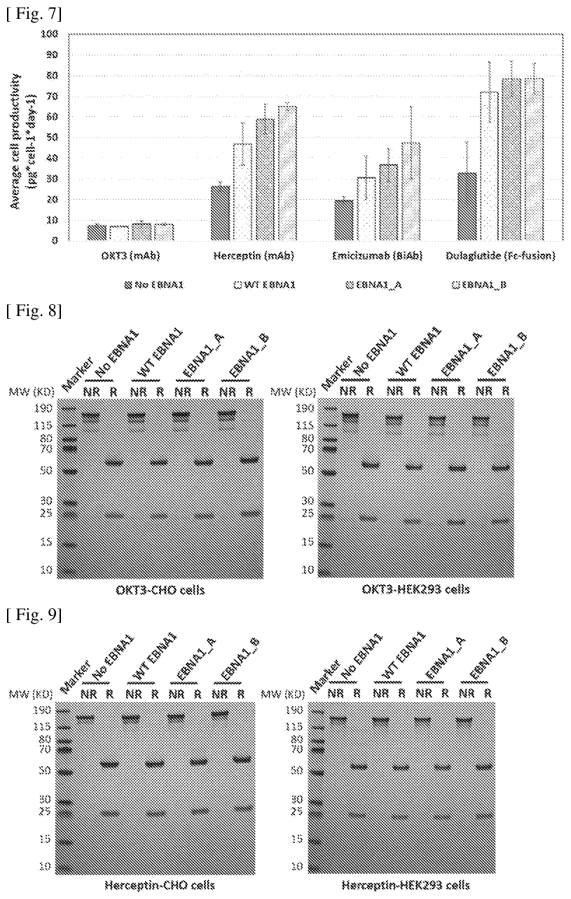

Four therapeutic molecules OKT3, Herceptin, Emicizumab and Dulaglutide, classified as monoclonal antibody (OKT3 and Herceptin), bispecific antibody (Emicizumab) and Fc-fusion protein (Dulaglutide), were applied to test the effects of engineered EBNA1s on their transient expression in CHO cells or HEK293 cells. Wild-type EBNA1, EBNA1_A and EBNA1_B variants can significantly enhance transient protein expression in mammalian cells to several folds higher. Moreover, EBNA1_A and EBNA1_B variants displayed better performance than wild-type EBNA1 in CHO cells. Especially in CHO cells transient transfection system, an average 1.8-fold titer increase has been achieved by EBNA1_A variant. In HEK293 cells transient transfection system, EBNA1_A also displayed slightly predominance effect on transient protein expression than wild-type EBNA1, while EBNA1_B was comparable to wild-type EBNA1 (FIG. 6 and FIG. 7). Thus, engineered EBNA1_A and EBNA1_B are preferable for CHO cells transient transfection system. In addition, stable cell lines constitutively expressing engineered EBNA1 were prepared to examine the effect of the cell lines on expression of proteins or polypeptides of interest. For an exemplary therapeutic protein, Adalimumab, significantly improved expression level was observed which verifies the effect of the stable cell line with engineered EBNA1 in the expression of proteins of interest.

Without wishing to be bound by theory, the long Glycine-Alanine rich linker (G-A repeat) of EBNA1 contains approximate 240 amino acid residues, and is highly dynamic, which may impair the translation efficiency. In addition, the dynamic of G-A repeat of EBNA1 may also negatively regulate its association efficiency to mitotic chromosomes. Thus, engineering of G-A repeat of EBNA1 may enhance its role in promoting recombinant protein expression. Moreover, EBNA1-chromosome association may relate to two basic regions containing AT hook DNA binding motif. AT hook is a conserved DNA binding motif, which exists in various chromosome proteins. Substitution of AT hook motifs of EBNA1 with AT hook motif of host chromosome-related proteins may enhanced EBNA1's role in maintaining exogenous OriP-containing plasmids in host cells. High mobility group (HMG) proteins may be essential for various chromosomal events, some of HMGs bearing AT hook specifically localize to chromosome. Hence, AT hook of HMG proteins are candidates for engineering AT hook of EBNA1.

Based on the above disclosures, we have provided novel mammalian cell expression systems having improved transient gene expression comprising vectors or stable cell lines expressing function-enhanced engineered EBNA1s, the preparation and use thereof.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements which do not affect the end result. The terms “comprising”, “comprises” and “comprised” may also include the term “consisting essentially of” and “consisting of”.

The phrase “consisting essentially of” indicates that the listed elements are required or mandatory, but that other elements are optional and may or may not be present depending upon whether or not they materially affect the activity or action of the listed elements. The phrase “consisting of” is meant to include, and is limited to, whatever follows the phrase “consisting of.” Thus, the phrase “consisting of” indicates that the listed elements are required or mandatory and that no other elements may be present.

The term “isolated” refers to a material that is substantially or essentially free from components that normally accompany it in its native state. The material can be a cell or a macromolecule such as a protein or nucleic acid. For example, an “isolated cell,” as used herein, refers to a cell, which has been purified from the cells in a naturally-occurring state.

### Engineered EBNA1s and Coding Nucleotide Molecules Thereof

As used herein, the term “wild-type” refers to the most common genotype of the gene (in the context of a gene) or the most common amino acid sequence found in nature (in the context of a polypeptide or protein), which can be part of a publicly accessible database (e.g., e.g., EMBL Nucleotide Sequence Database, NCBI Entrez, ExPasy, Protein Data Bank and the like). In the present disclosure, the amino acid sequence of the wild-type EBNA1 comprises the sequence of SEQ ID NO: 1. As used herein, the term “original” refers to a portion or whole of a wild-type molecule.

As used herein, the term “engineered” refers to an alteration in the normal sequence of a nucleic acid sequence or an amino acid sequence (e.g., a gene or a gene product), which may include manipulation of nucleic acid or polypeptide molecules by synthetic means (e.g. by recombinant techniques, in vitro peptide synthesis, by enzymatic or chemical coupling of peptides or some combination of these techniques). An engineered EBNA1 may refer to an analogue, fragment, derivative, or mutant which is derived from but different in structure and function from wild-type EBNA1, for example, expresses compounds, nucleic acids or proteins at levels that are not expressed by naturally occurring cells or organisms, preferably having enhanced EBNA1's function in recombinant protein production.

In some embodiments, the wild-type EBNA1 comprises, from N-terminal to C-terminal: B1-GA-B2-C, wherein B1 is a first basic domain; GA is a Glycine-Alanine rich domain; B2 is a second basic domain; C is the C-terminal region of EBNA1 comprising a nuclear localization signal (NLS), an OriP-DNA binding domain and an acidic domain.

In some embodiments, the wild-type EBNA1 comprises or consists of an amino acid sequence of SEQ ID NO: 1, wherein B1 comprises or consists of aa 33 to aa 84 of SEQ ID NO: 1; GA comprises or consists of aa 85 to aa 328 of SEQ ID NO: 1; B2 comprises or consists of aa 329 to aa 407 of SEQ ID NO: 1; C comprises or consists of aa 408 to aa 641 of SEQ ID NO: 1, and the OriP-DNA binding domain comprises or consists of aa 461 to aa 607 of SEQ ID NO: 1. In some embodiments, the wild-type EBNA1 is as shown in FIG. 1.

In some embodiments, wild-type EBNA1 is engineered by removing or replacing the G-A rich linker containing region. In some embodiments, the original G-A rich linker is replaced by a designed and engineered linker. In some embodiments, the engineered EBNA1 comprises, from N-terminal to C-terminal: B1-L-B2-C, wherein B1 is a first basic domain; L is an engineered linker; B2 is a second basic domain; and C is the C-terminal region of EBNA1 comprising a nuclear localization signal (NLS), an OriP-DNA binding domain and an acidic domain.

In some embodiments, the original G-A rich linker is replaced by a designed linker having an amino acid sequence of SEQ ID NO: 12 or having at least 80%, 85%, 90%, 95%, 98% or 99% sequence identity to SEQ ID NO: 12. In some embodiments, the variant has an amino acid sequence of SEQ ID NO: 2 or having at least 80%, 85%, 90%, 95%, 98% or 99% sequence identity to SEQ ID NO: 2. In some embodiments, the engineered EBNA1 is EBNA1_A as shown in FIG. 1.

In some embodiments, the original G-A rich linker-containing N terminal portion of wild-type EBNA1 is replaced by an AT hook motif derived from other proteins or species or by a modified AT hook motif. In some embodiments, the original N terminal portion of wild-type EBNA1 is replaced by an AT hook motif from high mobility group (HMG) proteins. In some embodiments, the AT hook motif for replacement is connected to the remaining portion of the wild-type EBNA1 directly or via a linker (such as a linker comprising less than 20, 18, 16, 14, 12, 10, 8, 6 amino acid residues).

In some embodiments, the engineered EBNA1 comprises, from N-terminal to C-terminal: AT-SL-C, wherein AT is an AT-hook motif; SL is a short linker; and C is the C-terminal region of EBNA1 comprising a nuclear localization signal (NLS), an OriP-DNA binding domain and an acidic domain.

In some embodiments, the original N terminal portion of wild-type EBNA1 is replaced by an AT hook motif and a short linker having an amino acid sequence of SEQ ID NO: 13 or having at least 80%, 85%, 90%, 95%, 98% or 99% sequence identity to SEQ ID NO: 13. In some embodiments, the variant has an amino acid sequence of SEQ ID NO: 3 or having at least 80%, 85%, 90%, 95%, 98% or 99% sequence identity to SEQ ID NO: 3. In some embodiments, the engineered EBNA1 is EBNA1_B as shown in FIG. 1.

In some embodiments, comparing to wild-type EBNA1, function-enhanced engineered EBNA1s significantly enhance protein production in mammalian cells.

Also provided herein is a nucleotide molecule encoding the engineered EBNA1 of the present disclosure. Preferably, the nucleotide molecule is codon optimized to improve the expression of the engineered EBNA1. The nucleotide molecule can be obtained by engineering the coding sequence of wild-type EBNA1 via conventionally used methods in the art or can be synthesized.

### Polypeptide of Interest

Any polypeptide of interest can be expressed in the mammalian cell according to the present disclosure. The term “polypeptide” refers to a molecule comprising a polymer of amino acids linked together by (a) peptide bond (s). Polypeptides include polypeptides of any length, including proteins (e.g. having more than 50 amino acids) and peptides (e.g. 2~49 amino acids). Polypeptides include proteins and/or peptides of any activity, function or size, and may include e.g. enzymes (e.g. kinases, phosphatases), receptors, transporters, bactericidal and/or endotoxin-binding proteins, structural polypeptides, membrane-bound polypeptides, glycopolypeptides, globular proteins, immune polypeptides, toxins, antibiotics, hormones, growth factors, blood factors, vaccines, viral glycopolypeptides and the like.

The polypeptide of interest that is expressed according to the teachings described herein may also be a subunit or domain of a polypeptide, such as e.g. a heavy chain or a light chain of an antibody or a functional fragment or derivative thereof. The term “polypeptide of interest” may refer to such individual subunit or domain or the final protein that is composed of the respective subunits or domains, depending on the context.

According to some embodiments, the polypeptide of interest is selected from a therapeutic or diagnostic polypeptide. Therapeutic and hence therapeutically active polypeptides are particularly important. The term therapeutic polypeptides also encompasses prophylactic polypeptides, e.g. used for vaccination. The polypeptide may be selected from the group consisting of peptide hormones, interleukins, tissue plasminogen activators, cytokines, growth factors, immunoglobulins, in particular antibodies or functional antibody fragments or variants or derivatives thereof and Fc-fusion proteins.

In some embodiments, the polypeptide of interest is an immunoglobulin molecule such as an antibody. The term “antibody” includes naturally occurring antibodies as well as all recombinant forms of antibodies, e.g., humanized antibodies, fully human antibodies and chimeric antibodies. Each heavy chain is usually comprised of a heavy chain variable region (VH) and a heavy chain constant region (CH). Each light chain is usually comprised of a light chain variable region (VL) and a light chain constant region (CL). The term “antibody”, however, also includes other types of antibodies such as single domain antibodies, heavy chain antibodies, i.e. antibodies only composed of one or more, in particular two heavy chains, and nanobodies, i.e. antibodies only composed of a single monomeric variable domain. Nanobodies may also be linked to form multivalent structures. As discussed above, the polynucleotide encoding the polypeptide of interest may also encode one or more subunits or domains of an antibody, e.g. a heavy or a light chain or a functional fragment or derivative thereof, as polypeptide of interest. Said subunits or domains can be expressed either from the same or different expression cassettes.

A “functional fragment or derivative” of an antibody in particular refers to a polypeptide which is derived from an antibody and is capable of binding to the same antigen, in particular to the same epitope as the antibody. It has been shown that the antigen-binding function of an antibody can be executed by fragments of a full-length antibody or derivatives thereof. Examples of fragments or derivatives of an antibody include (i) Fab fragments, monovalent fragments consisting of the variable region and the first constant domain of each the heavy and the light chain; (ii) F (ab) 2 fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv) 2 fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular.

According to the present disclosure, due to the presence of engineered EBNA1 (s) of the present disclosure in the expression system (such as a mammalian cell), the production of the polypeptide of interest is greatly enhanced.

### Expression Vectors

The molecules encoding engineered EBNA1 and/or polypeptide of interest of the present disclosed can be cloned into suitable vector (s) and introduced into a host cell for recombinant expression.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention may also comprise a selection marker, appropriate restriction sites to facilitate cloning of the desired gene and the ability to enter and/or replicate in eukaryotic or prokaryotic cells.

As known in the art, an expression vector can be used to introduce a heterologous polynucleotide into the host cell. The polynucleotides can be comprised in an expression cassette. The polynucleotide (s) encoding engineered EBNA1, the polypeptide of interest and optionally a selectable marker or reporter polypeptide may be located on the same or on different expression vectors. If they are located on different expression vectors, the expression vectors are co-transfected into the host cell. Such co-transfection strategies is well-known in the prior art. Introduction into a mammalian cell may be achieved e.g. by transfecting one or more suitable expression vectors comprising the polynucleotide encoding the polypeptide of interest and/or the engineered EBNA1 into the host cells.

Vectors suitable to be used in the present disclosure may comprise but not limit to a plasmid, a viral vector, a cosmid or an artificial chromosome. In some embodiments, for engineered EBNA1, the vector is an OriP-containing vector, such as a pWX4.1 plasmid. In some embodiments, the vector is a vector for constitutive expression of an engineered EBNA1, such as a pWX039 plasmid.

In case the heterologous nucleic acid is not inserted into the genome, the heterologous nucleic acid can be lost at the later stage e.g. when the cells undergo mitosis (for transient transfection). In some other aspects, the EBNA1_A or EBNA1_B expression vector may integrate into the genome of the host cell (for stable transfection). Stable transfection EBNA1_A or EBNA1_B cell line may be used for producing a polypeptide of interest.

Several appropriate methods are known in the prior art for introducing a heterologous nucleic acid such as an expression vector into mammalian host cells, and thus, do not need any detailed description herein. Respective methods include but are not limited to calcium phosphate transfection, electroporation, lipofection, biolistic- and polymer-mediated genes transfer and the like. Besides traditional random integration based methods also recombination mediated approaches can be used to transfer the heterologous polynucleotide into the host cell genome. As respective methods are well known in the prior art, they do not need any detailed description here. Non-limiting embodiments of suitable vector designs are also described subsequently and it is referred to the respective disclosure.

### Mammalian Cell and Recombinant Expression System

Also provided herein is a mammalian cell which can be used in high effective recombinant expression and a corresponding mammalian recombinant expression system.

The mammalian cell of the present disclosure may be selected from the group consisting of but not limit to rodent cells, human cells and monkey cells. Preferred mammalian cells are rodent cells such as e.g. cells derived from hamster or mouse. The rodent cell can be a cell line selected from the group consisting of a Chinese hamster cell line (such as e.g. a Chinese Hamster Ovary (CHO) cell line), a BHK cell line, a NS0 cell line, a C127 cell line, a mouse 3T3 fibroblast cell line, and a SP2/0 cell line. Particularly preferred is a CHO cell such as a CHO-K1 derived CHO cell.

In some embodiments, the mammalian cell is derived from a human cell, which may be e.g. selected from the group consisting of a HEK293 cell, a MCF-7 cell, a PerC6 cell, a CAP cell, hematopoietic cells and a HeLa cell. Another alternative are monkey cells, which, e.g., may be selected from the group consisting of a COS cells, COS-1, a COS-7 cell and a Vero cell.

According to some embodiment, the mammalian cell is provided as cell clone, cell line or cell culture. Host cell lines are typically available from commercial services, such as the American Tissue Culture Collection or from published literature.

### Pharmaceutical Compositions

The therapeutic or prophylactic proteins produced by the materials and methods of the present disclosed can be used to prepare pharmaceutical compositions for the treatment or prevention of human disease. A pharmaceutical composition of the present disclosure typically includes a therapeutically or prophylactically effective amount of a polypeptide of interest and a pharmacologically acceptable carrier.

As used herein, “pharmaceutically acceptable carrier” can be any solvent, dispersion medium, coating, antibacterial or antifungal agent, isotonic or absorption delaying agent, or the like. The use of such media and agents for pharmaceutically active substances is well known in the art. Supplementary active ingredients can also be incorporated into a pharmaceutical composition of the present invention.

Administration of a pharmaceutical composition of the present invention can be via any common route so long as the target tissue is available via that route. This includes oral, nasal, buccal, rectal, vaginal, or topical. Alternatively, administration can be by orthotopic, intradermal, subcutaneous, intramuscular, intraperitoneal, intratumoral, circumferentially, catheterization, or intravenous injection.

A pharmaceutical composition of the present disclosure can also be administered parenterally or intraperitoneally. Solutions of proteins of interest can be prepared in water suitably mixed with a surfactant, such as hydroxypropylcellulose. Dispersions can also be prepared in glycerol, liquid polyethylene glycols, or mixtures thereof, or in oils. Under ordinary conditions of storage and use, these preparations can contain a preservative to prevent the growth of microorganisms.

The pharmaceutical forms suitable for injectable use include sterile aqueous solutions or dispersions, or sterile powders for the extemporaneous preparation of sterile injectable solutions or dispersions. In many cases, the pharmaceutical forms are sterile and fluid to the extent that easy syringability exists. The pharmaceutical forms can also be stable under the conditions of manufacture and storage and preserved against the contaminating action of microorganisms, such as bacteria and fungi.

Suitable pharmaceutical carriers include, but are not limited to, solvents or dispersion media containing, for example, water, ethanol, polyol (e.g., glycerol, propylene glycol, and liquid polyethylene glycol, and the like), or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various antibacterial or antifungal agents, for example, parabens, chlorobutanol, phenol, sorbic acid, thimerosal, or the like. In many cases, it will be preferable to include isotonic agents, for example, sugars or sodium chloride. Prolonged absorption of the injectable compositions can be brought about by the use of agents delaying absorption, for example, aluminum monostearate and gelatin.

Sterile injectable solutions can be prepared by incorporating a therapeutic or prophylactic protein in the required amount in an appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients. In the case of sterile powders for the preparation of sterile injectable solutions, the preferred methods of preparation are vacuum-drying or freeze-drying techniques which yield a powder of the active ingredient plus any additional desired ingredient from a previously sterile-filtered solution thereof.

For oral administration, a therapeutic or prophylactic protein produced by the present invention can be incorporated with excipients and used in the form of non-ingestible mouthwashes and dentifrices. A therapeutic or prophylactic protein can also be dispersed in dentifrices, gels, pastes, powders, or slurries.

Upon formulation, compositions or solutions can be administered in a manner compatible with the dosage formulation and in such amount as is therapeutically or prophylactically effective. The dosage regimen can be determined by the attending physician based on various factors such as the action of the protein, the site of pathology, the severity of disease, the patient's age, sex and diet, the severity of any inflammation, time of administration, and other clinical factors. In one example, systemic or injectable administration is initiated at a dose which is minimally effective, and the dose is increased over a pre-selected time course until a positive effect is observed. Subsequently, incremental increases in dosage are made limiting to levels that produce a corresponding increase in effect while taking into account any adverse affects that may appear.

### Specific Embodiments

Further embodiments of the present invention are described again in the following. The present invention in particular also provides for the following items:

1. An engineered EBNA1, wherein as compared to wild-type EBNA1, the original G-A rich region is engineered in the engineered EBNA1.

2.1. The engineered EBNA1 of item 1, wherein
(a) the original G-A rich region is removed;
(b) the original G-A rich region is replaced by a linker; and/or
(c) the N-terminus fragment comprising the original G-A rich region (such as residue 1 to residue 336 at the N-terminus) of wild-type EBNA1 is replaced by an AT hook motif capable of binding to a chromosome at mitotic phase, such as an AT hook motif (such as residue 1 to residue 92) from human HMG-I/Y.

2.2. The engineered EBNA1 of item 1, wherein the wild-type EBNA1 comprises an amino acid sequence of SEQ ID NO: 1.

3. The engineered EBNA1 of item 1, wherein
the wild-type EBNA1 comprises, from N-terminal to C-terminal:
B1-GA-B2-C; and
the engineered EBNA1 comprises, from N-terminal to C-terminal:
(i) B1-L-B2-C; or
(ii) AT-SL-C,
wherein B1 is a first basic domain; GA is a Glycine-Alanine rich domain; B2 is a second basic domain; C is the C-terminal region of EBNA1 comprising a nuclear localization signal (NLS), an OriP-DNA binding domain and an acidic domain; L is an engineered linker; AT is an AT-hook motif; and SL is a short linker.

4.1. The engineered EBNA1 of item 2.1, wherein the engineered linker replacing the original G-A rich region has a length of 10-50, 20-40, 30-36 amino acids.

4.2. The engineered EBNA1 of item 2.1, wherein the engineered linker comprises the amino acid sequence of (RGRGGSGGGGSGGAGGGGSGGA)ₙGGSGGSGG, wherein n is 1, 2, 3, or 4.

4.3. The engineered EBNA1 of item 2.1, wherein the engineered linker comprises

(SEQ ID NO: 12)
RGRGGSGGGGSGGAGGGGSGGAGGSGGSGG.

5. The engineered EBNA1 of item 1, wherein
(a) the AT hook motif for replacement is linked to the original EBNA1 part via a short linker which has a length of less than 30, less than 25, less than 20 amino acid residues; and/or
(b) the short linker comprises an amino acid sequence of

(SEQ ID NO: 14)
GGGGAGGGGSGGGGAGGGGSGGGGAGGG;

(c) the AT hook motif comprises an amino acid sequence of

(SEQ ID NO: 15)
MSESSSKSSQPLASKQEKDGTEKRGRGRPRKQPPVSPGTALVGSQKE
PSEVPTPKRPRGRPKGSKNKGAAKTRKTTTTPGRKPRGRPKKLEK;

and/or
(d) the AT hook motif together with the short linker comprises an amino acid sequence of SEQ ID NO: 13.

6. The engineered EBNA1 of item 1, wherein the engineered EBNA1 comprises an amino acid sequence of SEQ ID NO: 2 or SEQ ID NO: 3.

7. An isolated polynucleotide molecule encoding the engineered EBNA1 of any of items 1-6.

8.1. The isolated polynucleotide molecule of item 7, wherein the polynucleotide molecule is in a form suitable for being introduced into a mammalian cell for recombinant expression of a polypeptide of interest.

8.2. The isolated polynucleotide molecule of item 7, wherein the engineered EBNA1 coding molecule is codon optimized.

9. A vector comprising one or more of the polynucleotide molecule of any of items 7-8.

10. The vector of item 9, wherein the vector is selected from a plasmid, a viral vector, a cosmid or an artificial chromosome.

11.1. The vector of item 9, wherein the vector is an OriP-containing vector, such as a pWX4.1 plasmid.

11.2. The vector of item 9, wherein the vector is further co-transfected with vector (s) containing OriP and/or coding molecules for a polypeptide of interest.

11.3. The vector of item 9, wherein the vector is for constitutive expression of the engineered EBNA1, such as a pWX039 plasmid.

12. An isolated mammalian cell suitable for recombinant expression of a polypeptide of interest, wherein the mammalian cell comprises an engineered EBNA1, wherein the engineered EBNA1 has enhanced function in recombinant expression as compared to wild-type EBNA1 (such as a wild-type EBNA1 comprises an amino acid sequence of SEQ ID NO: 1) in improving the recombinant expression, and wherein the original G-A rich region of wild-type EBNA1 is engineered in the engineered EBNA1.

13.1. The mammalian cell of item 12, wherein the original G-A rich region is:
(a) removed;
(b) replaced by an engineered linker; and/or
(c) the N-terminus fragment comprising the original G-A rich region (such as residue 1 to residue 336 at the N-terminus) of wild-type EBNA1 is replaced by an AT hook motif capable of binding to a chromosome at mitotic phase, such as an AT hook motif (such as residue 1 to residue 92) from human HMG-I/Y.

13.2. The mammalian cell of item 12, wherein the wild-type EBNA1 comprises an amino acid sequence of SEQ ID NO: 1.

14. The mammalian cell of item 12, wherein
the wild-type EBNA1 comprises, from N-terminal to C-terminal:
B1-GA-B2-C; and
the engineered EBNA1 comprises, from N-terminal to C-terminal:
(i) B1-L-B2-C; or
(ii) AT-SL-C,
wherein B1 is a first basic domain; GA is a Glycine-Alanine rich domain; B2 is a second basic domain; C is the C-terminal region of EBNA1 comprising a nuclear localization signal (NLS), an OriP-DNA binding domain and an acidic domain; L is an engineered linker; AT is an AT-hook motif; and SL is a short linker.

15.1. The mammalian cell of item 13, wherein the engineered linker replacing the original G-A rich region has a length of 10-50, 20-40, 30-36 amino acids.

15.2. The mammalian cell of item 13, wherein the engineered linker comprises the amino acid sequence of (RGRGGSGGGGSGGAGGGGSGGA)ₙGGSGGSGG, wherein n is 1, 2, 3, or 4.

15.3. The mammalian cell of item 13, wherein the engineered linker comprises

(SEQ ID NO: 12)
RGRGGSGGGGSGGAGGGGSGGAGGSGGSGG

16. The mammalian cell of item 13, wherein
(a) the AT hook motif for replacement is linked to the original EBNA1 part via a short linker which has a length of less than 30, less than 25, less than 20 amino acid residues; and/or
(b) the short linker comprises an amino acid sequence of

(SEQ ID NO: 14)
GGGGAGGGGSGGGGAGGGGSGGGGAGGG;

and/or
(c) the AT hook motif comprises an amino acid sequence of

(SEQ ID NO: 15)
MSESSSKSSQPLASKQEKDGTEKRGRGRPRKQPPVSPGTALVGSQKEPS
EVPTPKRPRGRPKGSKNKGAAKTRKTTTTPGRKPRGRPKKLEK ;

an/or
(d) the AT hook motif together with the short linker comprises an amino acid sequence of SEQ ID NO: 13.

17.1. The mammalian cell of item 12, wherein the engineered EBNA1 comprises an amino acid sequence of SEQ ID NO: 2 or SEQ ID NO: 3.

17.2. The mammalian cell of item 12, wherein the engineered EBNA1 is introduced into the mammalian cell by expression vectors comprising the coding molecule of the engineered EBNA1.

18. The mammalian cell of item 17, wherein
(a) the engineered EBNA1 coding molecule is codon optimized; and/or
(b) the vector is an OriP-containing plasmid; and/or
(c) the vector is a pWX4.1 plasmid or a pWX039 plasmid; and/or
(d) the vector further comprises a coding molecule for the polypeptide of interest; and/or
(e) the cell further comprises one or more separated vectors carrying a coding molecule for the polypeptide of interest; and/or
(f) the vector carrying engineered EBNA1 coding molecule is further co-transfected with vector (s) containing OriP and/or coding molecules for a polypeptide of interest; and/or
(g) the cell constitutively or transiently expressing the engineered EBNA1.

19. The mammalian cell of item 12, wherein the polypeptide of interest is selected from glycoproteins, antibodies, non-IgG proteins, Fc-fusion proteins, Fab fragments, protein complexes, peptidases, signal peptides, nanobodies, growth factors, hormones, cytokines, blood factors and enzymes, preferably a therapeutically active or diagnostic polypeptide.

20. The mammalian cell of item 12, wherein the polypeptide of interest is selected from: OKT3 (for example, comprising the amino acid sequence of SEQ ID NO: 4 and/or 5), Herceptin (for example, comprising the amino acid sequence of SEQ ID NO: 6 and/or 7), Emicizumab (for example, comprising the amino acid sequence of SEQ ID NO: 8, 9 and/or 10) and Dulaglutide (for example, comprising the amino acid sequence of SEQ ID NO: 11) or functional chains or fragments thereof.

21. The mammalian cell of item 12, wherein the mammalian cell is selected from a human cell or a rodent cell or a monkey cell.

22. The mammalian cell of item 12, wherein the mammalian cell has one or more of the following characters:
(a) the mammalian cell is a rodent cell derived from hamster or mouse; and/or
(b) the mammalian cell is a rodent cell line selected from the group consisting of a Chinese hamster cell line (such as e.g. a Chinese Hamster Ovary (CHO) cell line), a BHK cell line, a NS0 cell line, a C127 cell line, a mouse 3T3 fibroblast cell line, and a SP2/0 cell line; and/or
(c) the mammalian cell is derived from a human cell selected from the group consisting of a HEK293 cell (such as HEK293F-S), a MCF-7 cell, a PerC6 cell, a CAP cell, hematopoietic cells and a HeLa cell; and/or
(d) the mammalian cell is provided in form of a cell culture, cell line or cell clone.

23. A method for producing a mammalian cell according to any of items 12 to 22, comprising introducing into a mammalian cell the polynucleotide molecule of any of items 7-8 encoding a engineered EBNA1.

24. The method of item 23, wherein the polynucleotide molecule further encoding a polypeptide of interest, or a separated polynucleotide molecule encoding a polypeptide of interest is also introduced into the cell.

25. A method for recombinantly producing or increasing the recombinant expression yield of a polypeptide of interest, comprising
(a) culturing a mammalian cell of any of items 12-22 under conditions that allow for the expression of the polypeptide of interest;
(b) isolating the polypeptide of interest; and
(c) optionally, processing the isolated polypeptide of interest.

26. The method of item 25, wherein the polypeptide of interest is selected from glycoproteins, antibodies, non-IgG proteins, Fc-fusion proteins, Fab fragments, protein complexes, peptidases, signal peptides, nanobodies, growth factors, hormones, cytokines, blood factors and enzymes, preferably a therapeutically active or diagnostic polypeptide.

27. The method of item 25, wherein the polypeptide of interest is selected from: OKT3 (for example, comprising the amino acid sequence of SEQ ID NO: 4 and/or 5), Herceptin (for example, comprising the amino acid sequence of SEQ ID NO: 6 and/or 7), Emicizumab (for example, comprising the amino acid sequence of SEQ ID NO: 8, 9 and/or 10) and Dulaglutide (for example, comprising the amino acid sequence of SEQ ID NO: 11) or functional chains or fragments thereof.

28. A product for use in the recombinant expression of a polypeptide of interest comprising the isolated polypeptide of any of items 1-6, the polynucleotide molecule of any of items 7-8, the vector of any of items 9-11, and/or the mammalian cell of any of items 12-22.

29. A product comprising the polypeptide of interest produced using any of the isolated polypeptide of any of items 1-6, the polynucleotide molecule of any of items 7-8, the vector of any of items 9-11 and the mammalian cell of any of items 12-22, or produced by the method of any of items 25-27.

30. The product of item 29, wherein the product is selected from a medicament, a kit for diagnosis or detection, an enzyme formulation.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification and claims, the singular forms “a”, “an” and “the” include plural aspects unless the context clearly dictates otherwise. The terms “include,” “have,” “comprise” and their variants are used synonymously and are to be construed as non-limiting. Throughout the specification, where compositions are described as comprising components or materials, it is contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

The present invention is further illustrated by the following examples. These examples are provided merely for illustration purposes and shall not be interpreted to limit the scope or content of the present invention in any way.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### Examples

### Materials and Methods

### Protein Engineering

Based on the mechanism of EBNA1 in maintaining plasmids in mammalian cells, two strategies were employed to enhance the EBNA1 function, including replacing the G-A rich repeat of EBNA1 with a high-translational efficiency linker and replacing the original AT hook of EBNA1 with host AT hook specific for chromosome association. Two engineered EBNA1s (EBNA1_A and EBNA1_B) were designed.

The sequences of wild-type EBNA1, EBNA1_A and EBNA1_B are listed as follow:

Wild-type EBNA1 (SEQ ID NO: 1):
MSDEGPGTGPGNGLGEKGDTSGPEGSGGSGPQRRGGDNHGRGRGRGRGR
GGGRPGAPGGSGSGPRHRDGVRRPQKRPSCIGCKGTHGGTGAGAGAGGAGAG
GAGAGGGAGAGGGAGGAGGAGGAGAGGGAGAGGGAGGAGGAGAGGGAGA
GGGAGGAGAGGGAGGAGGAGAGGGAGAGGGAGGAGAGGGAGGAGGAGAG
GGAGAGGAGGAGGAGAGGAGAGGGAGGAGGAGAGGAGAGGAGAGGAGAG
GAGGAGAGGAGGAGAGGAGGAGAGGGAGGAGAGGGAGGAGAGGAGGAGA
GGAGGAGAGGAGGAGAGGGAGAGGAGAGGGGRGRGGSGGRGRGGSGGRGR
GGSGGRRGRGRERARGGSRERARGRGRGRGEKRPRSPSSQSSSSGSPPRRPPPG
RRPFFHPVGEADYFEYHQEGGPDGEPDVPPGAIEQGPADDPGEGPSTGPRGQGD
GGRRKKGGWFGKHRGQGGSNPKFENIAEGLRALLARSHVERTTDEGTWVAGV
FVYGGSKISLYNLRRGIALAIPQCRLTPLSRLPFGMAPGPGPQPGPLRESIVCYF
MVFLQTHIFAEVLKDAIKDLVMTKPAPTCNIRVTVCSFDDGVDLPPWFPPMVEG
AAAEGDDGDDGDEGGDGDEGEEGQE
EBNA1_A (SEQ ID NO: 2):
MSDEGPGTGPGNGLGEKGDTSGPEGSGGSGPQRRGGDNHGRGRGRGRGR
GGGRPGAPGGSGSGPRHRDGVRRPQKRPSCIGCKGTHGGTGAGARGRGGSGG
GGSGGAGGGGSGGAGGSGGSGGRGRGGSGGRGRGGSGGRRGRGRERARGGS
RERARGRGRGRGEKRPRSPSSQSSSSGSPPRRPPPGRRPFFHPVGEADYFEYHQE
GGPDGEPDVPPGAIEQGPADDPGEGPSTGPRGQGDGGRRKKGGWFGKHRGQG
GSNPKFENIAEGLRALLARSHVERTTDEGTWVAGVFVYGGSKTSLYNLRRGTA
LAIPQCRLTPLSRLPFGMAPGPGPQPGPLRESIVCYFMVFLQTHIFAEVLKDAIKD
LVMTKPAPTCNIRVTVCSFDDGVDLPPWFPPMVEGAAAEGDDGDDGDEGGDG
DEGEEGQE
EBNA1_B (SEQ ID NO: 3):
MSESSSKSSQPLASKQEKDGTEKRGRGRPRKQPPVSPGTALVGSQKEPSEV
PTPKRPRGRPKGSKNKGAAKTRKTTTTPGRKPRGRPKKLEKGGGGGGGGSG
GGGAGGGGSGGGGAGGGGRGRGEKRPRSPSSQSSSSGSPPRRPPPGRRPFFHPV
GEADYFEYHQEGGPDGEPDVPPGAIEQGPADDPGEGPSTGPRGQGDGGRRKKG
GWFGKHRGQGGSNPKFENIAEGLRALLARSHVERTTDEGTWVAGVFVYGGSK
TSLYNLRRGTALAIPQCRLTPLSRLPFGMAPGPGPQPGPLRESIVCYEMVFLQTH
IFAEVLKDAIKDLVMTKPAPTCNIRVTVCSFDDGVDLPPWFPPMVEGAAAEGD
DGDDGDEGGDGDEGEEGQE

### Plasmids Preparation

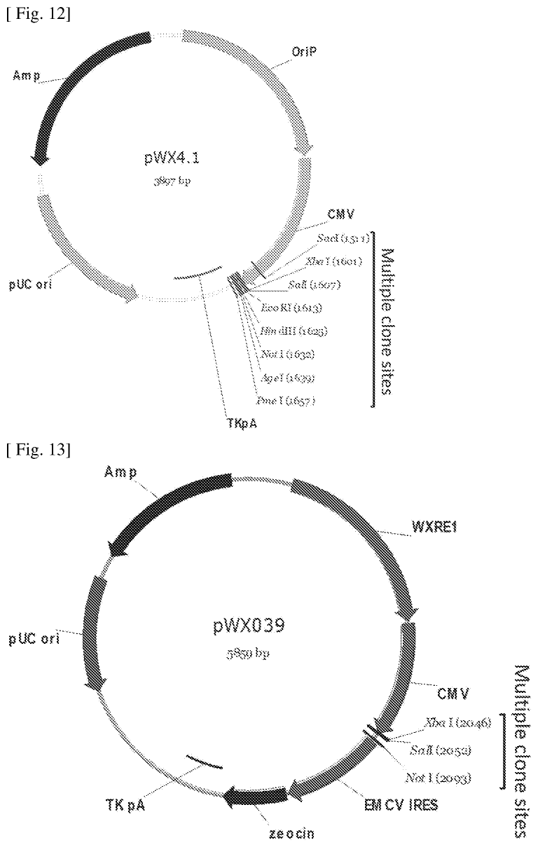

Genes encoding wild-type EBNA1 and engineered EBNA1s were codon optimized, synthesized (WuXi Biologics) and cloned into OriP-containing plasmids-pWX4.1 (WuXi Biologics), respectively. pWX4.1 plasmid is constructed upon pUC plasmid backbone containing Ori replication element (for plasmid replication in Escherichia coli) and ampicillin resistance gene (Amp for clone selection) by inserting multiple clone sites, CMV promoter and OriP element (FIG. 12). Genes encoding Wild-type EBNA1, EBNA1_A and EBNA1_B were cloned into SalI and NotI sites, respectively. Commercial antibodies and Fc-fusion proteins, including OKT3 (monoclonal antibody), Herceptin (monoclonal antibody), Emicizumab (Bispecific antibody) and Dulaglutide (Fc-fusion protein), were chosen to test the effect of EBNA1 and engineered EBNA1s. The protein sequences of test molecules were obtained from DrugBank. Genes encoding these antibodies and Fc-fusion protein were codon optimized, synthesized and cloned into pWX4.1 (WuXi Biologics), respectively. Genes encoding light chain and heavy chain of OKT3 were cloned into site between SalI and NotI respectively; genes encoding light chain and heavy chain of Herceptin were cloned into site between XbaI and NotI respectively; gene encoding light chain of Emicizumab was cloned into XbaI site; genes encoding heavy chains of Emicizumab was cloned into site between SalI and NotI respectively; and gene encoding Dulaglutide was cloned into site between SalI and NotI.

The protein sequences of test molecules are listed as follow:

OKT3 (DrugBank Accession Number: DB00075)
OKT3_light_chain (SEQ ID NO: 4):
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSGTKLE
INRADTAPTVSIFPPSSEQLTSGGASVVCFLNNFYPKDINVKWKIDGSERQNGVL
NSWTDQDSKDSTYSMSSTLTLTKDEYERHNSYTCEATHKTSTSPIVKSFNRNEC
OKT3_heavy_chain (SEQ ID NO: 5):
QVQLQQSGAELARPGASVKMSCKASGYTFTRYTMHWVKQRPGQGLEWI
GYINPSRGYTNYNQKFKDKATLTTDKSSSTAYMQLSSLTSEDSAVYYCARYYD
DHYCLDYWGQGTTLTVSSAKTTAPSVYPLAPVCGGTTGSSVTLGCLVKGYFPE
PVTLTWNSGSLSSGVHTFPAVLQSDLYTLSSSVTVTSSTWPSQSITCNVAHPASS
TKVDKKIEPRPKSCDKTHTCPPCPAPELLGGPSVFLFPPKPKDTLMISRTPEVTCV
VVDVSHEDPEVKENWYVDGVEVHNAKTKPREEQYNSTYRVVSVLTVLHQDW
LNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQG
NVFSCSVMHEALHNHYTQKSLSLSPGK
Herceptin (DrugBank Accession Number: DB00072)
Herceptin_light_chain (SEQ ID NO: 6):
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQGTKVEI
KRTVAAPSVFIFPPSDEQLKSGTASVVCLLNNFYPREAKVQWKVDNALQSQNS
QESVTEQDSKDSTYSLSSTLILSKADYEKHKVYACEVTHQGLSSPVTKSFNRGE
C
Herceptin _heavy_chain (SEQ ID NO: 7):
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSRWGGD
GFYAMDYWGQGTLVTVSSASTKGPSVFPLAPSSKSTSGGTAALGCLVKDYFPE
PVTVSWNSGALTSGVHTEPAVLQSSGLYSLSSVVTVPSSSLGTQTYICNVNHKPS
NTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQG
NVESCSVMHEALHNHYTQKSLSLSPGK
Emicizumab (DrugBank Accession Number: DB13923)
Emicizumab _light_chain (SEQ ID NO: 8):
DIQMTQSPSSLSASVGDRVTITCKASRNIERQLAWYQQKPGQAPELLIYQA
SRKESGVPDRFSGSRYGTDFTLTISSLQPEDIATYYCQQYSDPPLTFGGGTKVEIK
RTVAAPSVEIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Emicizumab _heavy_chain_1 (SEQ ID NO: 9):
QVQLVESGGGLVQPGGSLRLSCAASGFTFSYYDIQWVRQAPGKGLEWVSS
ISPSGQSTYYRREVKGRFTISRDNSKNTLYLQMNSLRAEDTAVYYCARRTGREY
GGGWYFDYWGQGTLVTVSSASTKGPSVFPLAPCSRSTSESTAALGCLVKDYFP
EPVTVSWNSGALTSGVHTFPAVLQSSGLYSLSSVVTVPSSSLGTQTYTCNVDHK
PSNTKVDKRVESKYGPPCPPCPAPEFLGGPSVFLFPPKPKDTLMISRTPEVTCVV
VDVSQEDPEVQFNWYVDGVEVHNAKTKPREEQYNSTYRVVSVLTVLHQDWL
NGKEYKCKVSNKGLPSSIEKTISKAKGQPREPQVYTLPPSQKEMTKNQVSLTCL
VKGFYPSDIAVEWESNGQPENNYKTTPPVLDSDGSFFLYSKLTVDKSRWQEGN
VFSCSVMHEALHNRYTQKSLSLSP
Emicizumab_heavy_chain_2 (SEQ ID NO: 10):
QVQLVQSGSELKKPGASVKVSCKASGYTFTDNNMDWVRQAPGQGLEWM
GDINTRSGGSIYNEEFQDRVIMTVDKSTDTAYMELSSLRSEDTATYHCARRKSY
GYYLDEWGEGTLVTVSSASTKGPSVFPLAPCSRSTSESTAALGCLVKDYFPEPV
TVSWNSGALTSGVHTFPAVLQSSGLYSLSSVVTVPSSSLGTQTYTCNVDHKPSN
TKVDKRVESKYGPPCPPCPAPEFLGGPSVFLFPPKPKDTLMISRTPEVTCVVVDV
SQEDPEVQFNWYVDGVEVHNAKTKPREEQYNSTYRVVSVLTVLHQDWLNGK
EYKCKVSNKGLPSSIEKTISKAKGQPREPQVYTLPPSQEEMTKNQVSLTCLVKG
FYPSDIAVEWESNGQPENNYKTTPPVLDSDGSFFLYSKLTVDKSRWQEGNVFSC
SVMHEALHNHYTQESLSLSP
Dulaglutide (DrugBank Accession Number: DB09045, SEQ ID NO: 11)
HGEGTFTSDVSSYLEEQAAKEFIAWLVKGGGGGGGSGGGGSGGGGSAES
KYGPPCPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHN
HYTQKSLSLSLG
Adalimumab (DrugBank Accession Number: DB00051)
Adalimumab_light_chain (SEQ ID NO: 16)
DIQMTQSPSSLSASVGDRVTITCRASQGIRNYLAWYQQKPGKAPKLLIYAA
STLQSGVPSRFSGSGSGTDFTLTISSLQPEDVATYYCQRYNRAPYTFGQGTKVET
KRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSENRGE
C
Adalimumab_heacy_chain (SEQ ID NO: 17)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAKVSYL
STASSLDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLIVLHQDW
LNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQG
NVFSCSVMHEALHNHYTQKSLSLSPGK

### Cell Culture, Transfection and Protein Expression

Above test molecules (including OKT3, Herceptin, Emicizumab and Dulaglutide) were transfected to CHO cells and HEK293 cells to verify the effect of EBNA1 and engineered EBNA1s, respectively. Suspension culture of CHO-K1 cells (ATCC) and HEK293F (ATCC) were performed at 36.5° C., 120 rpm and 6% CO₂ in a humidified orbital shaking incubator (Kuhner). CHO cells were maintained in CD CHO (Thermofisher) medium, and HEK293 cells were maintained in FreeStyle 293 Expression medium (Thermofisher). Transfection and protein expression were performed in 24 Deep-well plate (Kuhner).

Before CHO cell transfection, cells were washed and resuspended in Transpro CD01 (DUONING) to a final cell number of 10~16×10⁶ cells/ml, and 2.5 ml of suspended cells were plated into each well. Plasmids expressing test molecules were transfected alone or co-transfected with plasmids expressing wild-type EBNA1 or engineered EBNA1s by mixing with polyetherimide (BIOHUB) at DNA: PEI molar ratio of 1:2~1:5. After transfection, 5% CB7a (Hyclone), 0.5% CB7b (Hyclone) and Glucose were supplemented to medium on day 0 and day 4, and cultures were incubated at 225 rpm in incubator, and temperature was shifted to 31° C. on day 1.

For HEK293 cells transfection, cells were diluted to 2~-3×10⁶ cells/ml using Dynamis medium (Thermofisher) one day before transfection. When cell density reached 3~6×10⁶ cells/ml, 2.5 ml of cell culture was plated into each well of 24 Deep-well plate (Kuhner), and the transfection protocol was as described as above for CHO cell transfection. After transfection, feeding was performed on day 1 and day 3 by adding 5% CB7a (Hyclone), 0.5% CB7b (Hyclone) and Glucose.

Transfection of each molecule was repeated twice to ensure data integrity. CHO cells were harvested on day 7 and HEK293 cells were harvested on day 6, and cell viability was detected by Vi-CELL XR (Beckman).

### Protein Purification

Cell culture was harvested by centrifuge at 3000g, 4° C. for 30 min, supernatants was filtrated by 0.22 μM filter (Millipore). 200 μl filtrated supernatant of each well was subjected to titer determination, the remaining supernatant of each well was loaded on Tip column pre-packed with MabSelect SuRe (Cytiva) by using liquid workstation (Hamilton). Protein loaded tip columns were washed with wash buffer containing 25 mM sodium acetate, pH 6.0, recombinant proteins were eluted by 0.1 M sodium acetate pH 3.0, and final products were neutralized to pH 6.0 by adding 1M Tris pH 9.0. Samples were loaded and analyzed by SDS-PAGE.

### Concentration Determination of Fc-Containing Proteins and SDS-PAGE

Two hundred microliters (200 μl) filtrated supernatant from each well was collected, centrifuged and transferred into an Agilent 96-well plate (0.5 mL, round wells, U shape, 14 mm). Concentration of antibodies and Fc-fusion proteins were determined by protein A high-performance liquid chromatography on an Agilent 1260 Infinity II (Agilent Technologies) with a variable wavelength detector. Two mobile phases were prepared: (A) 50 mM sodium phosphate buffer (PB), 150 mM NaCl, pH 7.0; (B) 100 mM Glycine, 150 mM NaCl, pH 2.5. The standard (STD, human IgG4, Kappa) was first diluted using mobile phase A to 2.0 mg/mL. Antibodies or Fc-fusion protein in culture medium were quantified by comparing peak size with the standard curve.

One microgram (1 μg) of final products purified from CHO cell cultures and HEK293 cell cultures were loaded and analyzed by SDS-PAGE (SurePAGE, GenScript) to compare the product quality of each test molecules (including OKT3, Herceptin, Emicizumab and Dulaglutide).

### Example 1. Effects of Engineered EBNA1s on Protein Expression in CHO Cells

To test the function of engineered EBNA1s in CHO transient transfection system, wild-type EBNA1, EBNA1_A and EBNA1_B were co-expressed with test molecules respectively. The expression titer, cell viability, average cell productivity and final product quality were analyzed for each test molecule.

### 1.1 Effects of Engineered EBNA1s on OKT3 Expression in CHO Cells

The average expression titers for expressing OKT3 alone or co-expressing with EBNA1, EBNA1_A and EBNA1_B were 60 mg/L, 120 mg/L, 232 mg/L and 224 mg/L respectively (FIG. 2). Both engineered EBNA1s significantly increased OKT3 expression level in CHO cells. Furthermore, EBNA1_A and EBNA1_B enhanced OKT3 expression titer to almost two-fold higher than wild-type EBNA1.

We calculated the average cell productivity, and the result also indicated that EBNA1_A and EBNA1_B performed better than wild-type EBNA1 on recombinant OKT3 expression (FIG. 6).

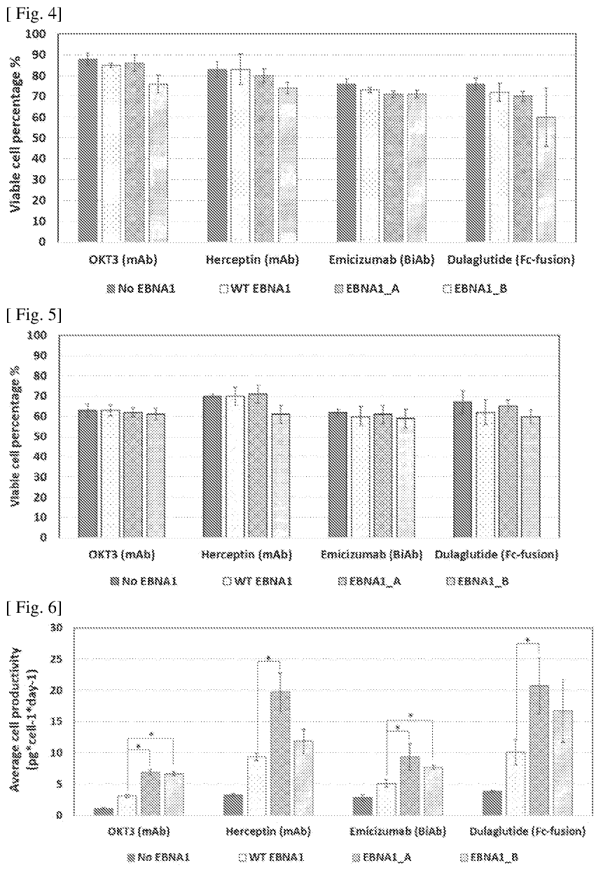

We also investigated the cytotoxic effects of EBNA1_A and EBNA_B, and the results showed that the cell viability of cell transfected with EBNA1_A and EBNA1_B were comparable to those of cells without EBNA1 transfection or transfected with wild-type EBNA1 (FIG. 4). SDS-PAGE analyses indicated a similar quality of final products (FIG. 8 left panel).

### 1.2 Effects of Engineered EBNA1s on Herceptin Expression in CHO Cells

The average expression titers for expressing Herceptin alone or co-expressing with EBNA1, EBNA1_A and EBNA1_B were 162 mg/L, 367 mg/L, 643 mg/L and 620 mg/L respectively (FIG. 2). Comparing to wild-type EBNA1, EBNA1_A and EBNA1_B exhibited ~1.8 fold titer enhancement on Herceptin expression in CHO cells.

Average cell productivity indicated a significant promotion of expression by EBNA1_A (FIG. 6).

EBNA1_A and EBNA1_B also displayed low cytotoxicity for Herceptin expression (FIG. 4). The final purified products showed a same quality on SDS-PAGE (FIG. 9 left panel).

### 1.3 Effects of Engineered EBNA1s on Emicizumab Expression in CHO Cells

The average expression titers for expressing Emicizumab alone or co-expressing with EBNA1, EBNA1_A and EBNA1_B were 110 mg/L, 180 mg/L, 330 mg/L and 333 mg/L respectively (FIG. 2). EBNA1_A and EBNA1_B performed better than wild-type EBNA1 for Emicizumab expression.

EBNA1_A and EBNA1_B also enhanced average cell productivity on this test molecule (FIG. 6).

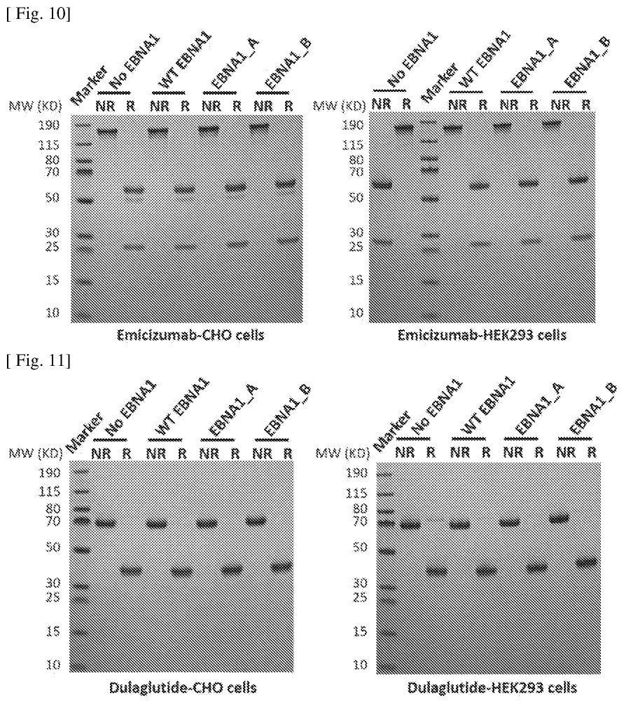

Viable cell analyses and SDS-PAGE analyses indicated that EBNA1_A and EBNA1_B displayed no side effects on cell viability and protein quality (FIG. 4 and FIG. 10 left panel).

### 1.4 Effects of Engineered EBNA1s on Dulaglutide Expression in CHO Cells

The average expression titers for expressing Emicizumab alone or co-expressing with EBNA1, EBNA1_A and EBNA1_B were 151 mg/L, 353 mg/L, 623 mg/L and 537 mg/L respectively (FIG. 2).

Similar to other test molecules, Dulaglutide expression was significantly enhanced by EBNA1_A and EBNA1_B.

The average cell productivity was also greatly enhanced by EBNA1_A (FIG. 6). In addition, EBNA1_A and EBNA1_B displayed low cytotoxicity and did not affect the protein quality (FIG. 4 and FIG. 11 left panel).

### Conclusion

The result indicated that EBNA1_A and EBNA1_B both perform better than wild-type EBNA1 in CHO cells. In addition, EBNA1_A and EBNA1_B displayed no significant cytotoxic on cell viability and side effects on protein quality.

### Example 2. Effects of Engineered EBNA1s on Protein Expression in HEK293 Cells

To test the function of engineered EBNA1s in HEK293 transient transfection system, wild-type EBNA1, EBNA1_A and EBNA1_B were co-expressed with test molecules respectively. The expression titer, cell viability, average cell productivity and final product quality were analyzed for each test molecule.

### 2.1 Effects of Engineered EBNA1s on OKT3 Expression in HEK293 Cells

The average expression titers for expressing OKT3 alone or co-expressing with EBNA1, EBNA1_A and EBNA1_B were 60 mg/L, 66 mg/L, 76 mg/L and 77 mg/L respectively (FIG. 3).

All engineered EBNA1s slightly enhanced OKT3 expression level in HEK293 cells. In addition, EBNA1_A and EBNA1_B performed slightly better than wild-type EBNA1. Average cell productivity analyses also indicated a slightly enhancement by EBNA1_A and EBNA1_B (FIG. 7).

No significant cytotoxic effect has been observed when expressing EBNA1_A and EBNA1_B (FIG. 5). Protein quality was also not affected by EBNA1_A and EBNA1_B (FIG. 8 right panel).

### 2.2 Effects of Engineered EBNA1s on Herceptin Expression in HEK293 Cells

The average expression titers for expressing Herceptin alone or co-expressing with EBNA1, EBNA1_A and EBNA1_B were 314 mg/L, 524 mg/L, 732 mg/L and 538 mg/L respectively (FIG. 3). EBNA1_A performed significantly better than EBNA1 and EBNA1_B were comparable to EBNA1. However, average cell productivity analyses suggested a better performance of EBNA1_A and EBNA1_B (FIG. 7).

Cell viability and protein quality were not affected by EBNA1_A and EBNA1_B (FIG. 5 and FIG. 9 right panel)

### 2.3 Effects of Engineered EBNA1s on Emicizumab Expression in HEK293 Cells

The average expression titers for expressing Emicizumab alone or co-expressing with EBNA1, EBNA1_A and EBNA1_B were 164 mg/L, 337 mg/L, 404 mg/L and 366 mg/L respectively (FIG. 3). EBNA1_A performed significantly better than EBNA1 and EBNA1_B were comparable to EBNA1. Average cell productivity enhancement by EBNA1_A and EBNA1_B were comparable to wild-type EBNA1 (FIG. 7).

Cell viability and protein quality were also not affected by EBNA1_A and EBNA1_B (FIG. 5 and FIG. 10 right panel) when expressing this test molecule.

### 2.4 Effects of Engineered EBNA1s on Dulaglutide Expression in HEK293 Cells

The average expression titers for expressing Dulaglutide alone or co-expressing with EBNA1, EBNA1_A and EBNA1_B were 321 mg/L, 727 mg/L, 946 mg/L and 702 mg/L respectively (FIG. 3). EBNA1_B displayed similar effect to wild-type EBNA1, while EBNA1_A worked much better than EBNA1. EBNA1_A and EBNA1_B shown a slightly dominant to EBNA1 (FIG. 7).

Cell viability and protein quality were not affected by EBNA1_A and EBNA1_B (FIG. 5 and FIG. 11 right panel) when expressing this test molecule.

### Conclusion

The results indicated that EBNA1_A displayed dominant effect on protein expression in HEK293 cells, while EBNA1_B is comparable to wild-type EBNA1 in HEK293 transient expression system. In addition, EBNA1_A and EBNA1_B displayed no significant cytotoxic on cell viability and side effects on protein quality.

### Example 3. Construction of Stable Cell Lines Constitutively Expressing Engineered EBNA1 and the Effect Thereof on Protein Production

To further investigate the effect of engineered EBNA1 on protein production of recombinant proteins of interest in mammalian cells, stable cell lines constitutively expressing engineered EBNA1 is prepared to examine the effect of the cell lines on expression of proteins or polypeptides of interest.

### 3.1 Construction of Stable Cell Lines Carrying Engineered EBNA1

Gene encoding engineered EBNA1 (EBNA1_B) was cloned into pWX039 plasmid (FIG. 13) (WuXi Biologics) for mammalian cell stable transfection. CHO cells were grown to a cell density of 1.5~2.0×10⁶ cells/ml, and 10⁷ cells were collected and resuspended in 300 μl electroporation buffer. Five g plasmids containing engineered EBNA1 gene were diluted into electroporation buffer and mixed with cells. Transfection was performed using Bio-Rad electroporator, and cells were transferred to pre-warmed CD CHO medium (Hyclone). Stable cells carrying engineered EBNA1 were selected by addition of 400 g/ml zeocin. Clones of stable cells expressing engineered EBNA1 were selected by limited dilution.

### 3.2 Expressing Protein of Interest Using Stable Cells Carrying Engineered EBNA1

Gene encoding protein of interest, Adalimumab was cloned into pWX4.1 vector and transfected into the above stable cell line. The cell line was further cultured under conditions allowing for the expression of the protein of interest.

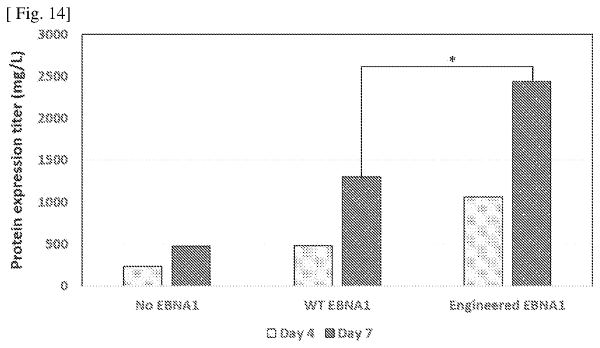

The results show that cell lines constitutively expressing engineered EBNA1 can be successfully constructed with significantly improved recombinant protein expression (about 1.8-fold increase of titer over cell line expressing wild type EBNA) (FIG. 14).

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

Information about the sequences in the Sequence Listing

| SEQ ID | |
|---|---|
| NO | INFORMATION |
| 1 | Wild-type EBNA (aa) |
| 2 | EBNA1_A (aa) |
| 3 | EBNA1_B (aa) |
| 4 | OKT3 light chain (aa) |
| 5 | OKT3 heavy chain (aa) |
| 6 | Herceptin light chain (aa) |
| 7 | Herceptin heavy chain (aa) |
| 8 | Emicizumab light chain (aa) |
| 9 | Emicizumab heavy chain-1 (aa) |
| 10 | Emicizumab heavy chain-2 (aa) |
| 11 | Dulaglutide (aa) |
| 12 | Engineered Linker in EBNA1_A (aa) |
| 13 | AT hook domain with short linker of |
| | EBNA1_B (aa) |
| 14 | Short linker in EBNA1_B (aa) |
| 15 | AT hook motif in EBNA1_B (aa) |
| 16 | Adalimumab light chain (aa) |
| 17 | Adalimumab heavy chain (aa) |

### REFERENCES

Nagesh, T., Ambuj, S., 2019. Recent Developments in Bioprocessing of Recombinant Proteins: Expression Hosts and Process Development. Front Bioeng Biotechnol. 7, 420.
Jianwei, Z., 2012. Mammalian cell protein expression for biopharmaceutical production. Biotechnol Adv. 30 (5), 1158-1170.
Kishwar, K., 2013. Gene expression in Mammalian cells and its applications. Adv Pharm Bull. 3 (2), 257-263.
Wolfgang, H., and Bill, S., 2013. Replication of Epstein-Barr viral DNA. Cold Spring Harb Perspect Biol. 5 (1), a013029.
John, S., Maki, U., Samantha, W., Christopher, O., Jaap, M., Ashok, A., 2004. The amino terminus of Epstein-Barr Virus (EBV) nuclear antigen 1 contains AT hooks that facilitate the replication and partitioning of latent EBV genomes by tethering them to cellular chromosomes. J Virol. 78 (21), 11487-11505.
Tohru, D., Ayumi, K., Masatoshi, F., Yutaka, S., Hiroki, I., Tatsuya, T., 2004. In vivo dynamics of EBNA1-oriP interaction during latent and lytic replication of Epstein-Barr virus. J Biol Chem. 279 (52), 54817-54825.
Ann, K., and Bill, S., 1995. Plasmid maintenance of derivatives of oriP of Epstein-Barr virus. J Virol. 69 (2), 1280-1283.
Francoise, L., Volker, P., Annie, B., Martine, T., Nathalie, S., Albine, B., Georg, S., Eric, J., Karola R., 1998. Up to 100-fold increase of apparent gene expression in the presence of Epstein-Barr virus oriP sequences and EBNA1: implications of the nuclear import of plasmids. J Virol. 72 (7), 6181-6185.
Olalekan, D., Jessica, S., Greg, D., Diane, H., Gary, P., William, H., Ray, F., 2014. A high-yielding CHO transient system: coexpression of genes encoding EBNA-1 and GS enhances transient protein expression. Biotechnol Prog. 30 (1), 132-141.
Joo-Hyoung, L., Sung-Min, L., Sun-Hye, P., Jeong-Ki, M., Gyun, L., Yeon-Gu, K., 2017. Investigation of relationship between EBNA-1 expression level and specific foreign protein productivity in transient gene expression of HEK293 cells. Process Biochem. 55, 182-186.

1. An engineered Epstein-Barr virus nuclear antigen 1 (EBNA1), wherein as compared to wild-type EBNA1, the original G-A rich region is engineered in the engineered EBNA1.

2. The engineered EBNA1 of claim 1, wherein
(a) the original G-A rich region is removed;
(b) the original G-A rich region is replaced by a linker; and/or
(c) the N-terminus fragment comprising the original G-A rich region (such as residue 1 to residue 336 at the N-terminus) of wild-type EBNA1 is replaced by an AT hook motif capable of binding to a chromosome at mitotic phase, such as an AT hook motif (such as residue 1 to residue 92) from human HMG-I/Y; and/or
wherein the wild-type EBNA1 comprises an amino acid sequence of SEQ ID NO: 1.

3. The engineered EBNA1 of claim 1, wherein
the wild-type EBNA1 comprises, from N-terminal to C-terminal:
B1-GA-B2-C; and
the engineered EBNA1 comprises, from N-terminal to C-terminal:
(i) B1-L-B2-C; or
(ii) AT-SL-C,
wherein B1 is a first basic domain; GA is a Glycine-Alanine rich domain; B2 is a second basic domain; C is the C-terminal region of EBNA1 comprising a nuclear localization signal (NLS), an OriP-DNA binding domain and an acidic domain; L is an engineered linker; AT is an AT-hook motif; and SL is a short linker.

4. The engineered EBNA1 of claim 2, wherein the engineered linker replacing the original G-A rich region has a length of 10-50, 20-40, 30-36 amino acids; and/or
wherein the engineered linker comprises the amino acid sequence of (RGRGGSGGGGSGGAGGGGSGGA)ₙGGSGGSGG, wherein n is 1, 2, 3, or 4; and/or
wherein the engineered linker comprises
(SEQ ID NO: 12)
RGRGGSGGGGSGGAGGGGSGGAGGSGGSGG.

5. The engineered EBNA1 of claim 1, wherein
(a) the AT hook motif for replacement is linked to the original EBNA1 part via a short linker which has a length of less than 30, less than 25, less than 20 amino acid residues; and/or
(b) the short linker comprises an amino acid sequence of
(SEQ ID NO: 14)
GGGGAGGGGSGGGGAGGGGSGGGGAGGG;
and/or
(c) the AT hook motif comprises an amino acid sequence of MSESSSKSSQPLASKQEKDGTEKRGRGRPRKQPP VSPGTALVGSQKEPSEV PTPKRPRGRPKGSKNKGAAKTRKTTTTPGRKPRGRPKKLEK (SEQ ID NO: 15); and/or
(d) the AT hook motif together with the short linker comprises an amino acid sequence of SEQ ID NO: 13.

6. The engineered EBNA1 of claim 1, wherein the engineered EBNA1 comprises an amino acid sequence of SEQ ID NO: 2 or SEQ ID NO: 3.

7. An isolated polynucleotide molecule encoding the engineered EBNA1 of any of claims 1-6.

8. The isolated polynucleotide molecule of claim 7, wherein the polynucleotide molecule is in a form suitable for being introduced into a mammalian cell for recombinant expression of a polypeptide of interest; and/or
wherein the engineered EBNA1 coding molecule is codon optimized.

9. A vector comprising one or more of the polynucleotide molecule of any of claims 7-8.

10. The vector of claim 9, wherein the vector is selected from a plasmid, a viral vector, a cosmid or an artificial chromosome.

11. The vector of claim 9, wherein the vector is an OriP-containing vector, such as a pWX4.1 plasmid; and/or
wherein the vector is further co-transfected with vector (s) containing OriP and/or coding molecules for a polypeptide of interest; and/or
wherein the vector is for constitutive expression of the engineered EBNA1, such as a pWX039 plasmid.

12. An isolated mammalian cell suitable for recombinant expression of a polypeptide of interest, wherein the mammalian cell comprises an engineered EBNA1, wherein the engineered EBNA1 has enhanced function in recombinant expression as compared to wild-type EBNA1 (such as a wild-type EBNA1 comprises an amino acid sequence of SEQ ID NO: 1) in improving the recombinant expression, and wherein the original G-A rich region of wild-type EBNA1 is engineered in the engineered EBNA1.

13. The mammalian cell of claim 12, wherein the original G-A rich region is:
(a) removed;
(b) replaced by an engineered linker; and/or
(c) the N-terminus fragment comprising the original G-A rich region (such as residue 1 to residue 336 at the N-terminus) of wild-type EBNA1 is replaced by an AT hook motif capable of binding to a chromosome at mitotic phase, such as an AT hook motif (such as residue 1 to residue 92) from human HMG-I/Y; and/or
the wild-type EBNA1 comprises an amino acid sequence of SEQ ID NO: 1.

14. The mammalian cell of claim 12, wherein
the wild-type EBNA1 comprises, from N-terminal to C-terminal:
B1-GA-B2-C; and
the engineered EBNA1 comprises, from N-terminal to C-terminal:
(i) B1-L-B2-C; or
(ii) AT-SL-C,
wherein B1 is a first basic domain; GA is a Glycine-Alanine rich domain; B2 is a second basic domain; C is the C-terminal region of EBNA1 comprising a nuclear localization signal (NLS), an OriP-DNA binding domain and an acidic domain; L is an engineered linker; AT is an AT-hook motif; and SL is a short linker.

15. The mammalian cell of claim 13, wherein the engineered linker replacing the original G-A rich region has a length of 10-50, 20-40, 30-36 amino acids; and/or
the engineered linker comprises the amino acid sequence of (RGRGGSGGGGSGGAGGGGSGGA)ₙGGSGGSGG, wherein n is 1, 2, 3, or 4; and/or
the engineered linker comprises RGRGGSGGGGSGGAGGGGS GGAGGSGGSGG (SEQ ID NO: 12).

16. The mammalian cell of claim 13, wherein
(a) the AT hook motif for replacement is linked to the original EBNA1 part via a short linker which has a length of less than 30, less than 25, less than 20 amino acid residues; and/or
(b) the short linker comprises an amino acid sequence of
(SEQ ID NO: 14)
GGGGAGGGGSGGGGAGGGGSGGGGAGGG;
and/or
(c) the AT hook motif comprises an amino acid sequence of MSESSSKSSQPLASKQEKDGTEKRGRGRPRKQPP VSPGTALVGSQKEPSEV PTPKRPRGRPKGSKNKGAAKTRKTTTTPGRKPRGRPKKLEK (SEQ ID NO: 15); and/or
(d) the AT hook motif together with the short linker comprises an amino acid sequence of SEQ ID NO: 13.

17. The mammalian cell of claim 12, wherein the engineered EBNA1 comprises an amino acid sequence of SEQ ID NO: 2 or SEQ ID NO: 3; and/or
wherein the engineered EBNA1 is introduced into the mammalian cell by expression vectors comprising the coding molecule of the engineered EBNA1.

18. The mammalian cell of claim 17, wherein
(a) the engineered EBNA1 coding molecule is codon optimized; and/or
(b) the vector is an OriP-containing plasmid; and/or
(c) the vector is a pWX4.1 plasmid or a pWX039 plasmid; and/or
(d) the vector further comprises a coding molecule for the polypeptide of interest; and/or
(e) the cell further comprises one or more separated vectors carrying a coding molecule for the polypeptide of interest; and/or
(f) the vector carrying engineered EBNA1 coding molecule is further co-transfected with vector (s) containing OriP and/or coding molecules for a polypeptide of interest; and/or
(g) the cell constitutively or transiently expressing the engineered EBNA1.

19. The mammalian cell of claim 12, wherein the polypeptide of interest is selected from glycoproteins, antibodies, non-IgG proteins, Fc-fusion proteins, Fab fragments, protein complexes, peptidases, signal peptides, nanobodies, growth factors, hormones, cytokines, blood factors and enzymes, preferably a therapeutically active or diagnostic polypeptide.

20. The mammalian cell of claim 12, wherein the polypeptide of interest is selected from: OKT3 (for example, comprising the amino acid sequence of SEQ ID NO: 4 and/or 5), Herceptin (for example, comprising the amino acid sequence of SEQ ID NO: 6 and/or 7), Emicizumab (for example, comprising the amino acid sequence of SEQ ID NO: 8, 9 and/or 10), Dulaglutide (for example, comprising the amino acid sequence of SEQ ID NO: 11), Adalimumab (for example, comprising the amino acid sequence of SEQ ID NO: 16 and/or 17), or functional chains or fragments thereof.

21. The mammalian cell of claim 12, wherein the mammalian cell is selected from a human cell or a rodent cell or a monkey cell.

22. The mammalian cell of claim 12, wherein the mammalian cell has one or more of the following characters:
(a) the mammalian cell is a rodent cell derived from hamster or mouse; and/or
(b) the mammalian cell is a rodent cell line selected from the group consisting of a Chinese hamster cell line (such as e.g. a Chinese Hamster Ovary (CHO) cell line), a BHK cell line, a NS0 cell line, a C127 cell line, a mouse 3T3 fibroblast cell line, and a SP2/0 cell line; and/or
(c) the mammalian cell is derived from a human cell selected from the group consisting of a HEK293 cell (such as HEK293F-S), a MCF-7 cell, a PerC6 cell, a CAP cell, hematopoietic cells and a HeLa cell; and/or
(d) the mammalian cell is provided in form of a cell culture, cell line or cell clone.

23. A method for producing a mammalian cell according to any of claims 12 to 22, comprising introducing into a mammalian cell the polynucleotide molecule of any of claims 7-8 encoding an engineered EBNA1.

24. The method of claim 23, wherein the polynucleotide molecule further encoding a polypeptide of interest, or a separated polynucleotide molecule encoding a polypeptide of interest is also introduced into the cell.

25. A method for recombinantly producing or increasing the recombinant expression yield of a polypeptide of interest, comprising
(a) culturing a mammalian cell of any of claims 12-22 under conditions that allow for the expression of the polypeptide of interest;
(b) isolating the polypeptide of interest; and
(c) optionally, processing the isolated polypeptide of interest.

26. The method of claim 25, wherein the polypeptide of interest is selected from glycoproteins, antibodies, non-IgG proteins, Fc-fusion proteins, Fab fragments, protein complexes, peptidases, signal peptides, nanobodies, growth factors, hormones, cytokines, blood factors and enzymes, preferably a therapeutically active or diagnostic polypeptide.

27. The method of claim 25, wherein the polypeptide of interest is selected from: OKT3 (for example, comprising the amino acid sequence of SEQ ID NO: 4 and/or 5), Herceptin (for example, comprising the amino acid sequence of SEQ ID NO: 6 and/or 7), Emicizumab (for example, comprising the amino acid sequence of SEQ ID NO: 8, 9 and/or 10) and Dulaglutide (for example, comprising the amino acid sequence of SEQ ID NO: 11) or functional chains or fragments thereof.

28. A product for use in the recombinant expression of a polypeptide of interest comprising the isolated polypeptide of any of claims 1-6, the polynucleotide molecule of any of claims 7-8, the vector of any of claims 9-11, and/or the mammalian cell of any of claims 12-22.

29. A product comprising the polypeptide of interest produced using any of the isolated polypeptide of any of claims 1-6, the polynucleotide molecule of any of claims 7-8, the vector of any of claims 9-11 and the mammalian cell of any of claims 12-22, or produced by the method of any of claims 25-27.

30. The product of claim 29, wherein the product is selected from a medicament, a kit for diagnosis or detection, an enzyme formulation.
